(19) United States
(12) Patent Application Publication
(10) Pub. No.: US 2023/0182137 A1
(43) Pub. Date: Jun. 15, 2023

(54) METHOD AND SYSTEM FOR CAR T-CELL SCREENING

(71) Applicant: Northwestern University, Evanston, IL (US)

(72) Inventors: Cheng Sun, Evanston, IL (US); Hao Zhang, Evanston, IL (US)

(21) Appl. No.: 18/080,067

(22) Filed: Dec. 13, 2022

Related U.S. Application Data
(60) Provisional application No. 63/288,784, filed on Dec. 13, 2021.

(51) Int. Cl. B01L 3/00

(52) U.S. Cl. CPC B01L 3/502761; B01L 3/502738; B01L 3/502715; B01L 2300/0816; B01L 2300/0867; B01L 2400/0487; B01L 2200/027

(57) ABSTRACT

A system for performing avidity-based screening includes a microfluidic circuit that has an inlet designed to receive target cells and CAR-T cells that bind to the target cells. A light source illuminates a portion of the microfluidic circuit that includes the CAR-T cells such that photoacoustic pressure is applied to the CAR-T cells. An imaging sensor captures images of the CAR-T cells. A processor is configured to analyze the images to determine if a CAR-T cell has detached from a target cell. Responsive to a determination that the CAR-T cell has detached, the processor records an amount of the photoacoustic pressure applied to the CAR-T cell. A desired avidity is determined based on a photoacoustic force spectrum, which is based on a plurality of photoacoustic pressures applied to a plurality of detached CAR-T cells. The processor also extracts one or more CAR-T cells that exhibit the desired avidity.

### CROSS-REFERENCE TO RELATED APPLICATION

The present application claims the priority benefit of U.S. Provisional Patent App. No. 63/288,784 filed on Dec. 13, 2021, the entire disclosure of which is incorporated herein by reference.

### STATEMENT REGARDING FEDERALLY SPONSORED RESEARCH

This invention was made with government support under grant numbers CBET1055379 and grant number DBI1353952 awarded by The National Science Foundation, and under grant number GM135018 awarded by The National Institutes of Health. The government has certain rights in this invention.

### BACKGROUND

Chimeric antigen receptor (CAR) T-cell therapy is a treatment used to fight cancer. T-cells are a type of white blood cell that is found in humans and that act as immune cells. In general, the human immune system identifies foreign substances in the body by finding proteins referred to as antigens on the surface of cells. T-cells also include proteins called receptors that attach to foreign antigens and help trigger other parts of the immune system to destroy the foreign substance. However, every different foreign antigen has a unique immune receptor that is able to bind to it. During a CAR T-cell therapy procedure, T-cells are taken from the blood of the patient, and a gene for a man-made receptor (the chimeric antigen receptor) is added to the T-cells such that the modified T-cells are able to more easily identify and bind to specific cancer cell antigens. The CAR T cells are then placed back into the patient so that they can begin binding with the cancer cells.

### SUMMARY

An illustrative system for performing avidity-based screening includes a microfluidic circuit that has an inlet designed to receive target cells and CAR-T cells that bind to the target cells. The system includes a light source configured to illuminate a portion of the microfluidic circuit that includes the CAR-T cells such that a photoacoustic pressure is applied to the CAR-T cells. An imaging sensor is configured to capture images of the CAR-T cells. A processor is operatively coupled to the light source and to the imaging sensor, and is configured to analyze the images to determine if a CAR-T cell has detached from a target cell. Responsive to a determination that the CAR-T cell has detached from the target cell, the processor records an amount of the photoacoustic pressure applied to the CAR-T cell. The processor also determines a desired avidity based at least in part on a photoacoustic force spectrum. The photoacoustic force spectrum is based on a plurality of photoacoustic pressures applied to a plurality of detached CAR-T cells. The processor also extracts, from the microfluidic circuit, one or more CAR-T cells that exhibit the desired avidity.

In an illustrative embodiment, responsive to the determination that the CAR-T cell has detached from the target cell, the processor is further configured to record a location of the CAR-T cell. The processor is further configured to determine whether all of the CAR-T cells have detached from the target cells, and the processor determines the photoacoustic force spectrum based on photoacoustic pressures applied to each of the CAR-T cells that detached from the target cells. Additionally, the extraction can occur responsive to a determination that all of the CAR-T cells have detached. In one embodiment, the imaging sensor comprises a complementary metal-oxide semiconductor camera or a charge-coupled device camera and the light source comprises a laser.

In another illustrative embodiment, the microfluidic circuit includes a main chamber in which the CAR-T cells bind to the target cells. The main chamber includes an array of photoacoustic transducers that, responsive to light from the light source, induce the photoacoustic pressure that is applied to the CAR-T cells. The microfluidic circuit also includes a sorting chamber into which the a plurality of detached CAR-T cells are guided. In another embodiment, the system includes an optically transparent ultrasound detector configured to determine pressure values of generated photoacoustic signals. In another embodiment, the processor is configured to tag the CAR-T cell that detaches from the target cell with location information of the CAR-T cell. The processor is also configured to tag the CAR-T cell that detaches from the target cell with the amount of the photoacoustic pressure that was applied to the CAR-T cell.

An illustrative method of performing avidity-based screening includes placing target cells into a microfluidic circuit along with CAR-T cells that bind to the target cells. The method includes illuminating, by a light source, a portion of the microfluidic circuit that includes the CAR-T cells such that a photoacoustic pressure is applied to the CAR-T cells. The method also includes capturing, by an imaging sensor, images of the CAR-T cells. The method also includes analyzing the images, by a processor operatively coupled to the light source and to the imaging sensor, to determine if a CAR-T cell has detached from a target cell. Responsive to a determination that the CAR-T cell has detached from the target cell, the method includes recording, by the processor, an amount of the photoacoustic pressure applied to the CAR-T cell. The method also includes determining, by the processor, a desired avidity based at least in part on a photoacoustic force spectrum, where the photoacoustic force spectrum is based on a plurality of photoacoustic pressures applied to a plurality of detached CAR-T cells. The method further includes extracting, by the processor and from the microfluidic circuit, one or more CAR-T cells that exhibit the desired avidity.

In one embodiment, placing the target cells in the microfluidic circuit comprises using a biochemical or physical agent to secure the target cells. In another embodiment, the target cells are placed such that a single target cell is attached each photoacoustic transducer in a plurality of photoacoustic transducers that are positioned relative to the microfluidic circuit. The method can also include recording, by the processor and responsive to the determination that the CAR-T cell has detached from the target cell, a location of the CAR-T cell. The method can also include tagging, by the processor, the detached CAR-T cell with the recorded amount of photoacoustic pressure. The method can also include determining, by an optically transparent ultrasound detector, pressure values of generated photoacoustic signals. The method can further include determining, by the processor, that all of the CAR-T cells have detached from the target cells, and determining the photoacoustic force spectrum based on photoacoustic pressures applied to each of the CAR-T cells that detached from the target cells.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts an avidity-based CAR-T cell screening system in accordance with an illustrative embodiment.

FIG. 2 depicts a magnified cross-sectional view of a photoacoustic transducer element from an array of photoacoustic transducers in accordance with an illustrative embodiment.

FIG. 3 depicts operations performed by an avidity-based CAR-T cell screening system in accordance with an illustrative embodiment.

FIG. 4 depicts a computing system for performing avidity-based screening of CAR-T cells in accordance with an illustrative embodiment.

### DETAILED DESCRIPTION

T-cell chimeric antigen receptors (CAR-T) can be used to screen and guide the T-cells of a patient to tumors cells expressing the cognate antigen. This therapeutic approach offers a unique solution in cancer therapy. However, in traditional CAR-T techniques, normal tissues with low expression of tumor-associated antigens (TAAs) can be mistargeted, resulting in severe side effects. To help prevent these side effects, a high throughput method to screen and select CAR-T cells that lead to minimal off-tumor and maximum antitumor functionalities in vitro is critical to warrant the efficacy of therapeutic outcomes in patients receiving CAR-T therapies.

In addition, it is desirable that the screening methodology used should introduce minimal or no perturbation to the physiological condition of the CAR-T cells themselves. The inventors have developed a device and associated methodologies to precisely examine how CAR-T cells bind to tumor cells in vitro as an effective way for screening, which is often referred to as avidity-based screening. However, traditional avidity-based screening methods are unable to generate accurately controllable force measurements. As a result, the existing avidity-based screening methods lack the capability of high throughput, lead to a high false-positive screening rate, and are unable to tailor screening for different cancers and patients.

Described herein are methods and systems that provide linearly tunable force for avidity-based screening. In an illustrative embodiment, the proposed methods and systems employ photoacoustic generation of focused unipolar ultrasound waves to facilitate precise spatial and temporal regulation of the force acting upon the T-cells, which enables significantly improved precision in the avidity-based screening process. Furthermore, photoacoustic generation can be readily parallelized, which allows high-throughput screening of up to 10⁸˜10⁹ T-cells. Alternatively, a different number of T-cells can be screened in the high-throughput screening process.

As discussed, the photoacoustic process is used to generate focused unipolar ultrasound signals. In one embodiment, photoacoustic generation of ultrasound employs a short pulse laser to illuminate light-absorbing materials. The composite and geometry of the light-absorbing materials can be configured to control the temporal profile of generated ultrasound waves after focusing. The transient thermo-expansion upon the absorption of the laser energy and the following rapid thermal relaxation collectively lead to a transient mechanical vibration of the light-absorbing materials. Such a vibration generates a temporally confined ultrasound pulse wave, referred to as photoacoustic wave, whose temporal profile is determined by the light-absorbing medium, the surrounding medium, and the illumination laser pulse duration. The photoacoustic process offers several unique advantages critical for developing a reliable avidity-based screen method.

The photoacoustic process allows for precise spatial control of the deposition of the acoustic energy. This precise control is an inherent advantage of using light to generate ultrasound signals as the light source (e.g., laser beam) can be readily steered and size-controlled to be from a millimeter scale to a sub-micrometer scale. Additionally, by controlling the geometry and composition of the light-absorbing materials, it is possible to coherently focus the generated ultrasound signal without the need for a focusing lens and either bipolar or unipolar temporal profile, a method commonly referred to as the beam-forming technique. These advantages are highly desirable for device integration and miniaturization. Collectively, using photoacoustic waves enables precise spatial control of the ultrasound energy distribution up to micrometer-scale, allowing avidity-based screening for individual CAR-T cells.

The photoacoustic process also allows for precise temporal profiling of the photoacoustically generated ultrasound radiation force. The photoacoustic signals are temporally confined, which provides sufficient temporal resolution to decipher the dynamic inter-cellular interactions. Furthermore, by controlling the constructive interference of the ultrasonic wave, it is possible to generate focused unipolar pressure pulses, which include a leading dominating positive pressure component followed by temporally stretched negative pressure with a much reduced peak pressure. Compared to the generation of a bipolar pressure wave using a piezoelectric transducer, the proposed unipolar pressure wave eliminates the unnecessary ambiguity of avidity testing due to the alternating positive and negative pressure acting of CAR-T cells. In addition, the proposed method uses lower total ultrasound energy to generate comparable force, minimizing the influence of the CAR-T cell viabilities.

Additionally, the photoacoustic process allows for wide dynamic range for precise force tuning. It has been established that the photoacoustic pressure is linearly proportional to the illuminating optical energy and material properties (e.g., optical absorption coefficient, thermal capacity, thermal relaxation coefficient, thermal conductivity, etc.). Thus, the precision of controlling the laser power and light absorbing material composite will precisely regulate the photoacoustic pressure. Furthermore, the photoacoustic process can generate a wide range of pressure from zero to the megapascal (MPa) range. The process therefore allows precise force tuning over a wide dynamic range in the avidity-based assay. The precise control and wide tuning range of the ultrasound force collectively enable the proposed method to be tailored for different cancers and patients.

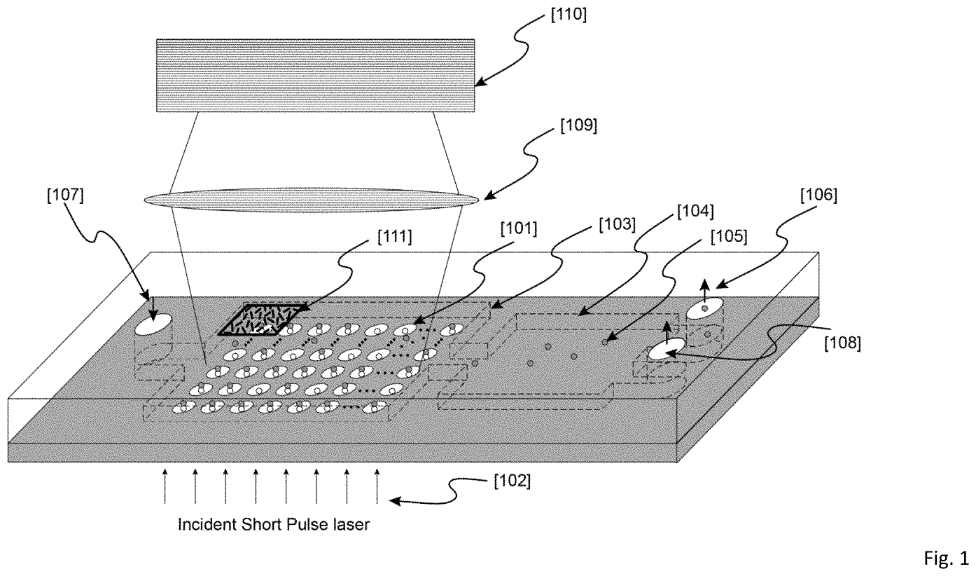

FIG. 1 depicts an avidity-based CAR-T cell screening system in accordance with an illustrative embodiment. As shown in FIG. 1, the avidity-based CAR-T cell screen system includes a large array of photoacoustic transducers 101 with matching cell anchoring cites. The system also includes an incident short-pulse laser 102 that is used to illuminate the bottom surface of the array of photoacoustic transducers 101 to generate a photoacoustic pressure onto the target cells. Any type of laser source may be used. In alternative embodiments, the system may include a different type of light source.

The system includes a layer of microfluidic circuits 103, which is integrated on the top of the array of photoacoustic transducers. A main fluidic chamber 103 covers the area of the array of photoacoustic transducers 101 to fulfill the function of cell seeding and collection. An additional microfluidic circuit chamber (or sorting chamber) 104 is cascaded to the main fluidic chamber 103 for sorting and enriching the lifted CAR-T cells 105, which can be sequentially collected through an outlet 106. A continuous flow of the fluid can be supplied through an inlet 107 that is fluidly connected to the outlet 108. The inlet 107 can also be used for cell seeding. In an alternative embodiment, the system may include a plurality of inlets 107, such as 2 inlets, 3 inlets, 4 inlets, etc. The top of the microfluidic circuit chamber 104 is designed to be optically transparent, allowing monitoring of the vertical motion of the T-cells under the given ultrasonic pressure using a top-mount imaging lens 109 and a matching imaging sensor array (e.g., a complimentary metal-oxide semiconductor (CMOS) camera, charge-coupled device (CCD) camera, etc.) 110. An optically transparent ultrasound detector 111 is integrated into the system to calibrate the true pressure value of the generated photoacoustic signals.

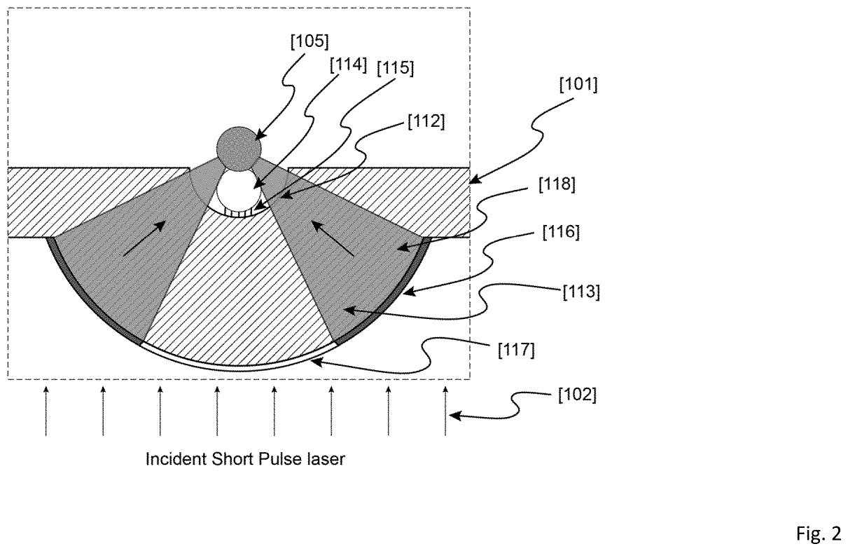

The detailed working principle is further explained with reference to FIG. 2. Specifically, FIG. 2 depicts a magnified cross-sectional view of a photoacoustic transducer element from the array of photoacoustic transducers 101 in accordance with an illustrative embodiment. The photoacoustic transducer includes a concave portion 112 on a top surface thereof, and a matching curved feature 113 on a bottom surface thereof. The concave portion 112 allows specific attachment of host cancer cells 114 using a biochemical or physical agent 115. The biochemical or physical agent 115 causes force interactions with the target cancer cells. Specifically, the biochemical or physical agent 115 is used to mount and/or immobilize the cancer cells onto the surface of concave portion 112 by establishing an attractive force interaction using either biochemical linkers or other forms of physical forces, such as a magnetic force, an optical force, an electrical force, etc.

Once the target cancer cells are immobilized on the surface of the concave portion 112, a CAR-T cell 105 will bind onto a host cancer cell 114. The size of the concave portion112 and the area of biochemical or physical agent 115 will be optimized only to allow a single host cancer (or tumor) cell 114/T cell 105 conjugate inside each of the concave portions 112. The curved feature 113 on the bottom is optimized for photoacoustic generations and host tumor cell housing. The outer surface of the curved feature 113 can be at least partially coated with a layer of light-absorbing material approximate to its circumference 116, while a center region can be coated with light-blocking materials 117. Such an arrangement allows two unique functions: 1) the arrangement shields the cancer cell 114 and the T cell 105 from the direct illumination of the short pulse laser 102; and 2) the arrangement provides focused acoustic waves 118 that propagate along the large angle of incidence and thus, preferentially act upon the T cell 105 with minimal interference from the cancer cell 114. In combining with the unique unipolar pressure at its focal point, the proposed arrangement can effectively exert pushing force to lift CAR-T cell 105 from the host cancer cell 114.

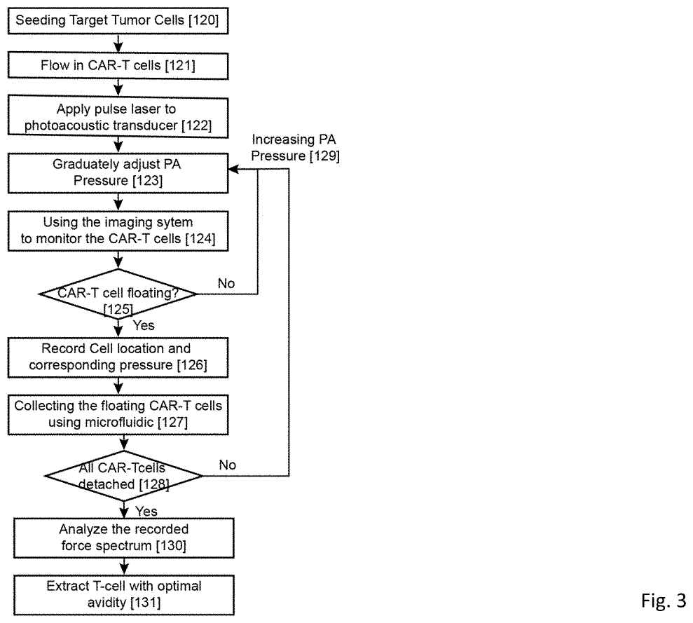

FIG. 3 depicts operations performed by an avidity-based CAR-T cell screening system in accordance with an illustrative embodiment. As shown in FIG. 3, the overall workflow of the screening process for the proposed system includes the following operations. In alternative embodiments, fewer, additional, and/or different operations may be performed. Also, the use of a flow diagram is not meant to be limiting with respect to the order of operations performed. In an operation 120, target cells are seeded on the surface of the chip via the inlet 107 of the microfluidic circuit. The target cell can be the cancer cell lines. Additional washing steps can be implemented to remove the unseeded cells.

In an operation 121, CAR-T cells are received via the inlet 107 and allowed to bind to the seeded target cells. Additional washing steps can be implemented to remove the unbonded T-cells. In an operation 122, a pulsed laser is used to illuminate the back surface of the chip, to generate focused photoacoustic waves at each of the T-cell 105 locations. In an operation 123, the photoacoustic pressure is adjusted by tuning the laser power. In an operation 124, concurrent to the operation 123, the system uses the top-mount imaging system to monitor the CAR-T cells being detached from the target cells In an operation 125, a determination is made regarding whether a CAR-T cell is being detached from a target cell. If it is determined that no CAR-T cell is being detached, then the system returns to the operation 123 and further increases the photoacoustic pressure. In an operation 126, if detached CAR-T cells are observed, the system records the location and the current photoacoustic pressure.

In an operation 127, the system collects the resulting floating detached T-cell using the microfluidic circuit. The system can also tag the collected floating T-cell with the recorded value(s) from the operation 126. In an operation 128, the system analyzes the recorded image using the imaging sensor array 110 to determine if all CAR-T cells are detached. If the system determines that there are remaining CAR-T cells, the system returns to the operation 123 and further increases the photoacoustic pressure. In an operation 130, if it is determined that all of the CAR-T cells are detached, the system records and analyzes the measured force spectrum, which can include the photoacoustic forces that resulted in detachment of each of the detached CAR-T cells. In an operation 131, the system determines the optimal avidity condition from the force spectrum obtained in the operation 130, and extracts T-cells being detached from the optimal condition.

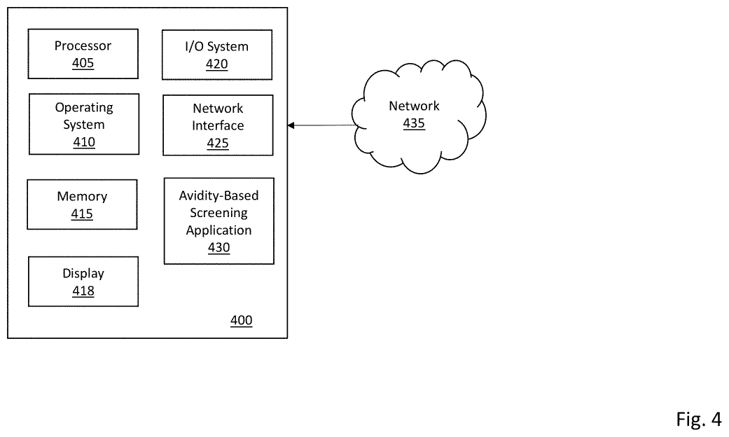

In an illustrative embodiment, any of the operations described herein can be performed by a computing system. As an example, FIG. 4 depicts a computing system 400 for performing avidity-based screening of CAR-T cells in accordance with an illustrative embodiment. In an illustrative embodiment, the computing system 400 can be incorporated into the avidity-based CAR-T cell screening system described with reference to FIG. 1. Alternatively, the computing system 400 can be separate from the avidity-based CAR-T cell screening system, but in communication therewith through a network 435 and/or through a direct wired connection.

The computing system 400 includes a processor 405, an operating system 410, a memory 415, a display 418, an input/output (I/O) system 420, a network interface 425, and an avidity-based screening application 430. In alternative embodiments, the computing system 400 may include fewer, additional, and/or different components. The components of the computing system 400 communicate with one another via one or more buses or any other interconnect system. The computing system 400 can be any type of computing system (e.g., smartphone, tablet, laptop, desktop, etc.), including a dedicated standalone computing system that is designed to perform the avidity-based screening.

The processor 405 can be in electrical communication with and used to control any of the system components described herein. For example, the processor can be used to execute the avidity-based screening application 430, control the imaging sensor, control cell delivery to the system, control cell removal from the system, perform mapping, display results, etc. The processor 405 can be any type of computer processor known in the art, and can include a plurality of processors and/or a plurality of processing cores. The processor 405 can include a controller, a microcontroller, an audio processor, a graphics processing unit, a hardware accelerator, a digital signal processor, etc. Additionally, the processor 405 may be implemented as a complex instruction set computer processor, a reduced instruction set computer processor, an x86 instruction set computer processor, etc. The processor 405 is used to run the operating system 410, which can be any type of operating system.

The operating system 410 is stored in the memory 415, which is also used to store programs, received measurements/data, network and communications data, peripheral component data, the avidity-based screening application 430, and other operating instructions. The memory 415 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 415 can be in the cloud to provide cloud storage for the system. Similarly, in one embodiment, any of the computing components described herein (e.g., the processor 405, etc.) can be implemented in the cloud such that the system can be run and controlled through cloud computing.

The I/O system 420 is the framework which enables users and peripheral devices to interact with the computing system 400. The display 418 can include a touch screen in some embodiments, and the touch screen can be part of the I/O system 420 that allows a user to make selections, control sub-systems, view results, etc. The display 418 can be any type of display, including a monitor, projector, etc., and can be used to present user interface screens, measured readings, and other data to the physician. The I/O system 420 can also include one or more speakers, one or more microphones, a keyboard, a mouse, one or more buttons or other controls, etc. that allow the user to interact with and control the computing system 400. The I/O system 420 also includes circuitry and a bus structure to interface with peripheral computing devices such as the imaging sensor, power sources, universal service bus (USB) devices, data acquisition cards, peripheral component interconnect express (PCIe) devices, serial advanced technology attachment (SATA) devices, high definition multimedia interface (HDMI) devices, proprietary connection devices, etc.

The network interface 425 includes transceiver circuitry (e.g., a transmitter and a receiver) that allows the computing system 400 to transmit and receive data to/from other devices such as remote computing systems, servers, websites, etc. The network interface 425 enables communication through the network 435, which can be one or more communication networks. The network 435 can include a cable network, a fiber network, a cellular network, a wi-fi network, a landline telephone network, a microwave network, a satellite network, etc. The network interface 425 also includes circuitry to allow device-to-device communication such as Bluetooth® communication.

The avidity-based screening application 430 can include software and algorithms in the form of computer-readable instructions which, upon execution by the processor 405, performs any of the various operations described herein such as seeding target cells on the surface of the microfluidic circuit, performing washing to remove unseeded target cells, introducing CAR-T cells for binding to the target cells, performing washing to remove unbonded T-cells, controlling the laser (or other light source) to illuminate the microfluidic circuit and generate photoacoustic waves at the T-cell locations, adjusting the photoacoustic pressure on the T-cells, controlling the top-mount imaging system to monitor the CAR-T cells being detached from the target cells, making a determination based on the imaging regarding whether CAR-T cells are being detached from the target cells, recording the location and the current photoacoustic pressure of detached T-cells, collecting the detached floating T-cells, tagging the collected T-cells with the recorded data, determining that all T-cells have been detached, recording and analyzing the measured force spectrum of the detached T-cells, determining the optimal avidity condition from the force spectrum, extracting T-cells that detached under the optimal condition, etc. The avidity-based screening application 430 can utilize the processor 405 and/or the memory 415 and/or the display 418 as discussed above. In an alternative implementation, the avidity-based screening application 430 can be remote or independent from the computing device 400, but in communication therewith.

As discussed above, any of the methods described herein can be performed by a computing system that includes a processor, a memory, a user interface, transceiver, etc. Any of the operations described herein can be stored in the memory as computer-readable instructions. Upon execution of these computer-readable instructions by the processor, the computing system performs the operations described herein.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to perform avidity-based screening, the system comprising:
a microfluidic circuit that includes an inlet designed to receive target cells and CAR-T cells that bind to the target cells;
a light source configured to illuminate a portion of the microfluidic circuit that includes the CAR-T cells such that a photoacoustic pressure is applied to the CAR-T cells;
an imaging sensor configured to capture images of the CAR-T cells; and
a processor operatively coupled to the light source and to the imaging sensor, wherein the processor is configured to:
analyze the images to determine if a CAR-T cell has detached from a target cell;
responsive to a determination that the CAR-T cell has detached from the target cell, record an amount of the photoacoustic pressure applied to the CAR-T cell;
determine a desired avidity based at least in part on a photoacoustic force spectrum, wherein the photoacoustic force spectrum is based on a plurality of photoacoustic pressures applied to a plurality of detached CAR-T cells; and
extract, from the microfluidic circuit, one or more CAR-T cells that exhibit the desired avidity.

2. The system of claim 1, wherein responsive to the determination that the CAR-T cell has detached from the target cell, the processor is further configured to record a location of the CAR-T cell.

3. The system of claim 1, wherein the processor is further configured to determine whether all of the CAR-T cells have detached from the target cells.

4. The system of claim 3, wherein the processor determines the photoacoustic force spectrum based on photoacoustic pressures applied to each of the CAR-T cells that detached from the target cells.

5. The system of claim 3, wherein the extraction occurs responsive to a determination that all of the CAR-T cells have detached.

6. The system of claim 1, wherein the imaging sensor comprises a complementary metal-oxide semiconductor camera or a charge-coupled device camera.

7. The system of claim 1, wherein the light source comprises a laser.

8. The system of claim 1, wherein the microfluidic circuit includes a main chamber in which the CAR-T cells bind to the target cells.

9. The system of claim 8, wherein the main chamber includes an array of photoacoustic transducers that, responsive to light from the light source, induce the photoacoustic pressure that is applied to the CAR-T cells.

10. The system of claim 1, wherein the microfluidic circuit also includes a sorting chamber into which the a plurality of detached CAR-T cells are guided.

11. The system of claim 1, further comprising an optically transparent ultrasound detector configured to determine pressure values of generated photoacoustic signals.

12. The system of claim 1, wherein the processor is configured to tag the CAR-T cell that detaches from the target cell with location information of the CAR-T cell.

13. The system of claim 1, wherein the processor is configured to tag the CAR-T cell that detaches from the target cell with the amount of the photoacoustic pressure that was applied to the CAR-T cell.

14. A method of performing avidity-based screening, the method comprising:
placing target cells into a microfluidic circuit along with CAR-T cells that bind to the target cells;
illuminating, by a light source, a portion of the microfluidic circuit that includes the CAR-T cells such that a photoacoustic pressure is applied to the CAR-T cells;
capturing, by an imaging sensor, images of the CAR-T cells;
analyzing the images, by a processor operatively coupled to the light source and to the imaging sensor, to determine if a CAR-T cell has detached from a target cell;
responsive to a determination that the CAR-T cell has detached from the target cell, recording, by the processor, an amount of the photoacoustic pressure applied to the CAR-T cell;
determining, by the processor, a desired avidity based at least in part on a photoacoustic force spectrum, wherein the photoacoustic force spectrum is based on a plurality of photoacoustic pressures applied to a plurality of detached CAR-T cells; and
extracting, by the processor and from the microfluidic circuit, one or more CAR-T cells that exhibit the desired avidity.

15. The method of claim 14, wherein the placing the target cells in the microfluidic circuit comprises using a biochemical or physical agent to secure the target cells.

16. The method of claim 15, wherein the target cells are placed such that a single target cell is attached each photoacoustic transducer in a plurality of photoacoustic transducers that are positioned relative to the microfluidic circuit.

17. The method of claim 14, further comprising recording, by the processor and responsive to the determination that the CAR-T cell has detached from the target cell, a location of the CAR-T cell.

18. The method of claim 14, further comprising tagging, by the processor, the detached CAR-T cell with the recorded amount of photoacoustic pressure.

19. The method of claim 14, further comprising determining, by an optically transparent ultrasound detector, pressure values of generated photoacoustic signals.

20. The method of claim 14, further comprising:
determining, by the processor, that all of the CAR-T cells have detached from the target cells; and
determining, by the processor, the photoacoustic force spectrum based on photoacoustic pressures applied to each of the CAR-T cells that detached from the target cells.
